(19) United States
(12) Patent Application Publication
(10) Pub. No.: US 2022/0404338 A1
(43) Pub. Date: Dec. 22, 2022

(54) IN VITRO CELL BASED POTENCY ASSAY

(71) Applicants: Peter A. DEPHILLIPS, West Point, PA (US); Nisarg M. PATEL, West Point, PA (US); Jingyuan XU, West Point, PA (US); Zhi-Qiang ZHANG, West Point, PA (US); Merck Sharp & Dohme LLC, Rahway, NJ (US)

(72) Inventors: Peter A. DePhillips, Collegeville, PA (US); Nisarg M. Patel, Feasterville-Trevose, PA (US); Jingyuan Xu, Berwyn, PA (US); Zhi-Qiang Zhang, Lansdale, PA (US)

(73) Assignee: Merck Sharp & Dohme LLC, Rahway, NJ (US)

(21) Appl. No.: 17/779,427

(22) PCT Filed: Nov. 30, 2020
(86) PCT No.: PCT/US2020/062539

§ 371 Date: May 24, 2022

Related U.S. Application Data
(60) Provisional application No. 62/942,878, filed on Dec. 3, 2019.

(51) Int. Cl. G01N 33/50

(52) U.S. Cl. CPC G01N 33/5023; G01N 33/5044

(57) ABSTRACT

The present disclosure provides an in vitro cell based potency assay to determine the relative potency of a composition, including a pharmaceutical composition, comprising an mRNA encapsulated in a lipid nanoparticle (LNP) as compared to a reference sample. Also provided is a process for releasing or accepting a batch of a pharmaceutical composition comprising an mRNA encapsulated in an LNP using the in vitro cell based potency assay. The methods and processes described comprise (i) transfecting a population of cells with a test sample of the composition, (ii) transfecting a different population of cells with a reference sample of the pharmaceutical composition, wherein the cells in step (ii) are the same cell type as the cells in step (i); (iii) detecting the amount of expression of a polypeptide encoded by the mRNA in the transfected cells; and comparing the amount of expression, thereby determining the relative in vitro potency of the composition.

### CROSS REFERENCE TO RELATED APPLICATIONS

### Field of the Invention

The present disclosure provides an in vitro cell based potency assay to determine the relative potency of a pharmaceutical composition comprising an mRNA encapsulated in a lipid nanoparticle (LNP) as compared to a reference sample.

### BACKGROUND OF THE INVENTION

Messenger RNA (mRNA)-based therapeutics are of great interest in the field of vaccines as safe and efficient alternatives to traditional live virus or protein-based vaccines (Kramps, T., Probst, J., RNA 2013, 4(6) 737-749). Unlike traditional vaccines, mRNA can be engineered to carry specific genetic information, which can be directly injected and delivered into cells where the antigen (i.e., the expression product of the mRNA) is generated in vivo. Lipid nanoparticles (LNPs) are used as delivery mechanisms for such mRNAs, where the mRNA is encapsulated within the LNP (mRNA-LNPs). In-vivo transfection and subsequent protein translation of mRNA delivered via an LNP is a multi-step process, with cell-entry by endocytosis, followed by escape of mRNA from the endosomal membrane vesicle into the cytosol, binding of the mRNA to ribosomes, and subsequent translation of protein.

RNA is not stable and can undergo degradation during preparation, process, formulation and storage. Physical and chemical properties of an LNP or of an mRNA can change over time and potentially impact the potency of an mRNA-LNP, either by impacting the ability of the LNP to be taken up into cells or the level of subsequent expression by such cells of the mRNA contained therein. Characterization of RNA and mRNA-LNPs is crucial to quality assurance. Therefore, reliable analytical methods that measure such characteristics of uptake and subsequent expression are required. Establishment of such an analytical method to assess mRNA stability and potency is valuable in assessing, for example, whether a manufactured batch of an mRNA-LNP pharmaceutical composition is suitable to release to the public or whether a production failure has occurred which impacts the cellular uptake of the mRNA-LNP or impacts expression of the mRNA. Described herein is an in vitro cell-based potency assay to determine the relative activity, stability and/or potency of a pharmaceutical composition comprising an mRNA encapsulated in a lipid nanoparticle (LNP) as compared to a reference sample.

### BRIEF SUMMARY OF THE INVENTION

Provided herein are methods for determining the relative in-vitro potency of a composition comprising an mRNA encapsulated in a lipid nanoparticle (LNP). The methods comprise (i) transfecting a population of cells with a test sample of the composition, (ii) transfecting a different population of cells with a reference sample of the pharmaceutical composition, wherein the cells in step (ii) are the same cell type as the cells in step (i); (iii) detecting the amount of expression of a polypeptide encoded by the mRNA in the transfected cells of steps (i) and (ii); and (iv) comparing the amount of expression of the polypeptide determined for the test sample in step (iii) with the amount of expression of the polypeptide determined for the reference sample in step (iii), thereby determining the relative in vitro potency of the composition. In one embodiment, the cells in step (i) are selected from Vero cells, HeLa cells, RD cells, Hep-2 cells and Hep-G2 cells.

In some embodiments, the detection of the expression of the polypeptide in step (iii) comprises contacting the transfected cells with a first antibody specific for the polypeptide encoded by the mRNA and subsequently with a second, labeled antibody which is specific for the first antibody. In further embodiments, the method further comprising detecting the second, labeled antibody. In some embodiments, the detection of the second, labeled antibody comprises measuring the fluorescence of the second, labeled antibody.

In some embodiments, the cells in step (i) are Vero cells, Hela cells, Hep-2 cells or RD cells. In additional embodiments, the method further comprises adding ApoE during the transfection step. In additional embodiments, ApoE is added in an amount of 4 μg/mL.

In some embodiments, no ApoE is added during the transfection step. In certain embodiments where no ApoE is added during the transfection step, the cells are Hep-G2 or RD cells. In specific embodiments, the cells are Hep-G2 cells.

In some embodiments, the LNP comprises a cationic lipid, a sterol, a non-cationic lipid and a peglyated-lipid.

In an embodiments of any of the above methods, the method further comprises seeding the cells on a cell culture plate comprising at least 12, 24, 48, 96 or 384 wells prior to transfecting the cells. In some embodiments, the wells of the cell culture plate do not contain a coating. In other embodiments, the wells of the cell culture plate are coated. In certain embodiments where the wells of the cell culture plate are coated, the coating is collagen or lysine.

In some embodiments, the seeded cells are grown to a confluency in which a monolayer of cells is formed. In some embodiments, the seeded cells are grown for about 16 to about 32 hours prior to transfecting. In other embodiments, the seeded cells are grown for about 20 to about 28 hours prior to transfecting.

In some embodiments, the cell culture plate has 96 wells. In some embodiment, the cell culture plate is a 96 well culture plate and each well of the cell culture plate is seeded with about 1.1×105 cells to about 1.4×105 cells per well when the wells of the cell culture plate are not coated. In certain embodiments, each well of the cell culture plate is seeded with 1.2×105 cells per well.

In some embodiments, the cell culture plate is a 96 well culture plate and each well is coated, for example, with collagen or lysine. In some embodiments the cells are Hep-G2 cells and the cells are seeded in a 96 well plate at a density of 15,000 cells per well to 35,000 cells per well when each well of the cell culture plate is coated. In additional embodiments, each well is coated with 20,000 cells per well to 30,000 cells per well. In further embodiments, each well of the cell culture plate is seeded with 30,000 cells per well.

In some embodiments of the above methods, the transfecting process of step (i) occurs at 35-39° C. with 4-6% CO2 for at least 4 hours.

In embodiments of any of the above, the method comprises generating a dose response curve for the test sample and the reference sample and determining the EC50 of the test sample and the reference sample. In some embodiments of the method, the relative potency is calculated as a percentage of the reference standard EC50 using the formula

(EC50 reference standard/EC50test sample)*100

Also provided herein is a process for releasing or accepting a batch of a pharmaceutical composition comprising an mRNA encapsulated in an LNP using the methods described herein. The process comprises (i) determining the relative in vitro potency of a test sample of the pharmaceutical composition from the batch according to any of the methods as described above; and (ii) releasing further pharmaceutical compositions from the batch for in vivo use if the results of step (i) indicate an acceptable relative in vitro potency value.

In some embodiments of the process, the relative in vitro potency value is calculated by generating a dose response curve for the test sample and the reference sample and determining the EC50 of the test sample and reference sample. In certain embodiments of the process, the relative in vitro potency value is calculated using the formula

(EC50 reference standard/EC50test sample)*100

In still further embodiments, the acceptable relative in vitro potency value is calculated to be between 50% and 200%.

In any of the above embodiments, the mRNA which is encapsulated within the LNP may be an mRNA encoding an RSV F peptide or a VZV glycoprotein.

### BRIEF DESCRIPTION OF THE DRAWINGS

FIG. 1 sets forth a general schematic of the in-vitro cell-based potency assay.

FIGS. 2A-2D set forth the transfection and protein translation for an mRNA encapsulated in an LNP for various cell-types tested. FIG. 2A shows the percentage of transfected cells on the Y axis and the corresponding mRNA (ng) on the X axis for HepG2, Vero, A549 and ARPE-19 cells. FIG. 2B shows the percentage of transfected cells on the Y axis and the corresponding mRNA (ng) on the X axis for HepG2 and Hep-2 cells. FIG. 2C shows the percentage of transfected cells on the Y axis and the corresponding mRNA (ng) on the X axis for Raw 264.7 cells, HeLa cells and Caco-2 cells. FIG. 2D shows the percentage of transfected cells on the Y axis and the corresponding mRNA (ng) on the X axis for HepG2 and RD cells.

FIGS. 3A and 3B show the total protein fluorescence on the Y axis for a given dose of mRNA (ng) on the X axis. FIG. 3A shows results for cells grown to 90-100% confluency. FIG. 3B shows the results for the cells grown to 75% confluency.

FIGS. 4A, 4B and 4C show percentage of transfected cells (normalized on the Y axis at various dosages of mRNA (ng) on the X-axis.

FIG. 5 shows the percentage of transfected cells on the Y axis at various dosages of mRNA (ng) on the X axis at seeding densities of 15K cpw, 20K cpw, 25K cpw, 30K cpw and 35K cpw when the tissue culture plate is coated with collagen.

FIG. 6 shows the percentage of transfected cells on the Y axis at various dosages of mRNA (ng) on the X axis after a transfection duration of 4 hours, 6 hours, 8 hours or 16 hours when the tissue culture plate is coated with collagen.

FIG. 7 shows percentage of transfected cells (normalized on the Y axis at various dosages of mRNA (ng) on the X-axis.

### DETAILED DESCRIPTION OF THE INVENTION

Described herein is an in-vitro cell-based potency assay to determine the potency of, or monitor the potency over time of, a pharmaceutical composition containing an LNP encapsulating an mRNA (mRNA-LNP). Physical and chemical properties of an LNP or of an mRNA can change over time and potentially impact the potency of an mRNA-LNP, either by impacting the ability of the LNP to be taken up into cells or the level of subsequent expression by such cells of the mRNA contained therein. Monitoring the potency of a pharmaceutical composition containing an LNP encapsulating an mRNA using the in-vitro cell-based potency assay described herein indicates if there are changes to the LNP and/or changes to the mRNA that impact potency of the pharmaceutical composition containing the mRNA-LNP.

In an embodiment, provided herein is a method for detecting the in vitro expression of an expression product (e.g., a polypeptide) encoded by an mRNA of a pharmaceutical composition comprising the mRNA encapsulated in an LNP, the method comprising transfecting a population of cells with the pharmaceutical composition and detecting the expression product encoded by the mRNA of the pharmaceutical composition in the transfected cells. In some embodiments, the cells are Vero cells, HeLa cells, RD cells, Hep-2 cells or Hep-G2 cells. In some embodiments, the cells are Hep-G2 cells.

In one embodiment, provided herein is a method for determining the relative in-vitro potency of a pharmaceutical composition comprising an mRNA encapsulated in a lipid nanoparticle (LNP), the method comprising: (i) transfecting a population of cells with a test sample of the pharmaceutical composition comprising the mRNA encapsulated in the LNP; (ii) transfecting a different population of cells with a reference sample of the pharmaceutical composition; (iii) detecting expression of a peptide encoded by the mRNA in the transfected cells of step (i) and step (ii); and (iv) comparing the expression of the peptide determined for the test sample in step (iii) with the expression of the peptide determined for the reference sample in step (iii) thereby determining the relative in vitro potency of the pharmaceutical composition. In some embodiments, the cells are Vero cells, HeLa cells, RD cells, Hep-2 cells or Hep-G2 cells. In some embodiments, the cells in step (i) and step (ii) are HepG2 cells.

In some embodiments, the method is performed on a series of dilutions of the test sample of the pharmaceutical composition and a series of dilutions of the reference sample of the pharmaceutical composition and a dose-response curve for each of the test samples and reference samples is generated as described herein. The dose responses curves of the test and reference samples can then be compared to determine the relative potency of the test sample of the pharmaceutical composition. In certain embodiments, the relative in vitro potency is calculated by comparing the EC50 of the test sample and the reference sample. In some embodiments, the relative in vitro potency is calculated using the formula:

(EC50 reference standard/EV50test sample)*100

In one embodiment, the detection of the peptide expressed by the mRNA comprises contacting the transfected cells of steps (i) and (ii) with a first antibody specific to the peptide encoded by the mRNA. In another embodiment, the detection further comprises subsequently contacting the transfected cells of step (i) and (ii) with a second, labeled antibody which is specific for the first antibody. In one embodiment, the second antibody is fluorescently labeled. For example, the second, labeled antibody can be IRDye 680 RD goat α-human antibody or Alexa Fluor 488 goat α-human antibody. The second, labeled antibody is typically specific to the species of the first antibody. In a further embodiment, the detection comprises detecting the second labeled antibody by measuring the fluorescence of the second, labeled antibody.

Generally, the LNPs of the compositions used in the assay described herein are composed of one or more cationic lipids (including ionizable cationic lipids) and one or more poly(ethyleneglycol)-lipids (PEG-lipids). In some embodiments, the LNP comprises a cationic lipid (including an ionizable cationic lipid), a sterol, a non-cationic lipid and a PEG-lipid. In some embodiments the sterol is cholesterol or a derivative thereof. Examples of non-cationic lipids include phospholipid-related materials, such as natural phospholipids, synthetic phospholipids, synthetic phospholipid derivatives, fatty acids, sterols, and combinations thereof. In further embodiments, the LNP comprises a cationic lipid (including an ionizable cationic lipid), cholesterol, a phospholipid and a PEG-lipid.

In one embodiment, the method further comprises, prior to transfecting the population of cells, seeding a population of cells on a cell culture plate. In one embodiment, the cell culture plate comprises at least 6, 12, 24, 48, 96, 384 or 1536 wells. In one embodiment, the plate comprises at least 12 wells. In another embodiment, the plate comprises at least 24 wells. In another embodiment, the plate comprises at least 48 wells. In another embodiment, the plate comprises at least 96 wells. In another embodiment, the plate comprises at least 384 wells. In another embodiment, the plate comprises at least 1536 wells. In one embodiment, the plate is a 96 well plate.

In one embodiment of the method, the wells of the cell culture plate do not contain a coating. In another embodiment, the wells of the cell culture plate are coated. In a further embodiment, the wells of the cell culture plate are coated with collagen or lysine. In one embodiment, the cells are coated with collagen. In another embodiment, the cells are coated with lysine.

In one embodiment of the method, prior to transfection, the seeded cells are grown to a confluency in which a monolayer of cells is formed. In some embodiments, the growth time of the seeded cells prior to transfection is up to 32 hours. In another embodiment, the growth time of the seeded cells prior to transfection is about 16 to about 32 hours. In one embodiment, the growth time is about 20 to about 28 hours prior to transfection. In a further embodiment, the growth time is about 16, 17, 18, 19, 20, 21, 22, 23, 24, 25, 26, 27, 28, 29, 30, 31, or about 32 hours prior to transfection.

In embodiments where the cell culture plate is a 96 well plate and the cell culture plate is not coated, the method further comprises seeding the cell culture plate with about 1.1×10⁵ cells to about 1.4×10⁵ cells per well. In another embodiment, the cell culture plate is seeded with 1.2×10⁵ cells per well.

In embodiments where the cell culture plate is a 96 well plate and the cell culture plate is coated, the method further comprises seeding the cell culture plate at a density of about 15,000 cells per well to 35,000 cells per well. In one embodiment, the wells of the cell culture plate are seeded with 20,000 cells per well to 30,000 cells per well. In other embodiment, the wells of the cell culture plate are seeded with about 20,000 cells per well. In another embodiment, the wells are seeded with 30,000 cells per well.

In any of the above embodiments, the transfection of step (i) and step (ii) occurs at 35-39C, with 4-6% CO₂ for at least 4 hours. In another embodiment, the duration of the transfection is up to 48 hours. In a further embodiment, the duration of the transfection is for about 14 hours to about 18 hours. In one embodiment, the duration of the transfection is 14 hours, or at least 14 hours. In another embodiment, the duration of the transfection is 15 hours, or at least about 15 hours. In one embodiment, the duration of the transfection is 16 hours, or at least about 16 hours. In an embodiment, the duration of the transfection is 17 hours, or at least about 17 hours. In a further embodiment, the duration of transfection is 18 hours, or at least about 18 hours.

Also provided herein is a process for releasing or accepting a batch of a pharmaceutical composition comprising an mRNA encapsulated in an LNP, the process comprising determining the relative in vitro potency of a test sample of the pharmaceutical composition from the batch according to any of the embodiment described above, and releasing further a process for analyzing a batch of a pharmaceutical composition comprising an mRNA encapsulated in an LNP, comprising determining the relative in vitro potency of a test sample of the pharmaceutical composition from the batch according to the method described above; and releasing further pharmaceutical compositions from the batch for in vivo use if the results determined relative in vitro potency of the test sample indicate an acceptable relative in vitro potency value.

In some embodiments, the process is performed on a series of dilutions of the test sample of the pharmaceutical composition from the batch and a series of dilutions of the reference sample of the pharmaceutical composition and a dose-response curve for each of the test samples and reference samples is generated as described herein. The dose responses curves of the test and reference samples can then be compared to determine the relative potency of the test sample of the pharmaceutical composition. In certain embodiments, the relative in vitro potency is calculated by comparing the EC50 of the test sample and the reference sample. In some embodiments, the relative in vitro potency is calculated using the formula:

(EC50 reference standard/EV50test sample)*100

In one embodiment of the above process, the acceptable relative in vitro potency value is between 50% and 200%. In another embodiment, the acceptable relative in vitro potency is 50% or greater.

In any of the above methods or process, the cells in step (i) and step (ii) are the same cell type. In certain embodiments, the cells are Vero cells, HeLa cells, RD cells, Hep-2 cells or Hep-G2 cells. In some embodiments, ApoE is added to the media. In some embodiments, it is added to the media at the transfection step. The addition of ApoE to certain cell lines allows for complete transfections. In other embodiments, no ApoE is added to the media. In some embodiments, the cells in step (i) and step (ii) are HepG2 cells. In some embodiments, the cells are HepG2 cells and no ApoE is added to the media.

In some embodiments of any of the foregoing, the mRNA is an mRNA encoding an RSV F protein, as described, for example, in WO 2017/070622 (PCT/US2016/058321), WO 2018/170260 (PCT/US2018/022630) or WO 2019/148101 (PCT/US2019/015412), the contents of each of which are incorporated by reference in their entirety. In other embodiments of any of the foregoing, the mRNA is an mRNA encoding a VZV glycoprotein, as described, for example, in WO/2017/070601 (PCT/US2016/058297), the contents of which are hereby incorporated by reference in their entirety.

### I. Definitions and Abbreviations

As used throughout the specification and appended claims, the following abbreviations apply:

APC: Antigen Presenting Cell

ApoE: Apolipoprotein E

CPW: Cells per well

EMEM: Eagle's minimal essential medium

FBS: Fetal Bovine Serum

LNP: Lipid nanoparticle

mRNA: Messenger RNA

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

Reference to “or” indicates either or both possibilities unless the context clearly dictates one of the indicated possibilities. In some cases, “and/or” was employed to highlight either or both possibilities.

As used herein, including the appended claims, the singular forms of words such as “a,” “an,” and “the,” include their corresponding plural references unless the context clearly dictates otherwise.

The term “about”, when modifying the quantity of a substance, the pH of a solution/formulation, or the value of a parameter characterizing a step in a method, or the like refers to variant in the numerical quantity that can occur, for example, through typical measuring, handling and sampling procedures involved in the preparation, characterization and/or use of the substance or composition; through inadvertent error in these procedures, through differences in the manufacture, source or purity of the ingredients employed to make or use the compositions or carryout the procedures and the like. In certain embodiments, “about” can mean a variation of greater or lesser than the value or range of values stated by 10 percent, e.g., ±0.2%, 0.5%, 1%, 2%, 3%, 4%, 5%, or 10%. Each value or range of values preceded by the term “about” is also intended to encompass the embodiment of the stated absolute value or range of values.

“Comprising” or variations such as “comprise”, “comprises” or “comprised of” are used throughout the specification and claims in an inclusive sense, i.e., to specify the presence of the stated features but not to preclude the presence or addition of further features that may materially enhance the operation or utility of any of the embodiments of the invention, unless the context requires otherwise due to express language or necessary implication.

“Consists essentially of,” and variations such as “consist essentially of” or “consisting essentially of,” as used throughout the specification and claims, indicate the inclusion of any recited elements or group of elements, and the optional inclusion of other elements, of similar or different nature than the recited elements, that do not materially change the basic or novel properties of the specified composition or method.

“Encapsulated” as used herein refers to the process or result of confining one or more agents, e.g., mRNA, within a lipid nanoparticle.

“Expression” as used herein refers to the biological process(es) that results in production of a polypeptide from a nucleic acid sequence, such as an mRNA sequence.

“Transfection” as used herein refers to the introduction of a nucleic acid, e.g., mRNA, into a cell. In one embodiment of the in-vitro cell-based potency assay described herein, cells are transfected with an LNP sample encapsulating an mRNA.

“Transfection duration”, “duration of transfection”, or “transfection time” each as used herein, refers to the amount of time cells are incubated after the mRNA-LNP is added to the cells seeded in the wells. In one embodiment, transfection time or transfection duration is 48 hours or less. In an embodiment of the present invention, the transfection time is 24, 23, 22, 21, 20, 19, 18, 17, 16, 15, 14, 13, 12, 11, 10, 9, 8, 7, 6 or 5 hours or less. In another embodiment, the transfection time is 16 hours. In one embodiment, the transfection time is from 5 hours to 48 hours. In another embodiment, the transfection time is 7 hours. In a further embodiment, the transfection time is between 5-20 hours, 5-16 hours, 7-20 hours or 7-16 hours.

“In-Vitro Potency” or “potency” as used herein refers to a measure of the ability of the transfected cells to express the product (e.g., a polypeptide) encoded by the mRNA that is encapsulated in the LNP in the cell-based potency assay described herein. In one embodiment, in-vitro potency is expressed as the “EC50” which represents the dose at which the value of the measured response (e.g., expression of the peptide or protein encoded by the mRNA) is halfway between the background and the maximum response. This midpoint can be determined by generating a dose response curve and fitting a four-parameter logistics regression model for the dose response curve. In an embodiment, the response measured is the percentage of cells per well which are expressing the peptide expressed by the mRNA encapsulated in the LNP. In another embodiment, the response measured is the total protein fluorescence per well.

“Relative in-vitro potency” or “relative potency” as used herein is the comparison of the potency of a test sample to the potency of a reference standard. For example, when potency is determined at the EC50, the relative potency of the test sample is calculated as a percentage of the reference standard EC50 as follows:

(EC50 reference standard/EC50test sample)*100

“Dose response curves” generated using the cell-based potency assay described herein refer to the dose of the mRNA LNP and the response refers to the measured parameter (e.g., expression) that corresponds to such dose.

“Test Sample” or “Test Article” as used herein refers to an aliquot of material obtained from a source of interest, such as, for example, a pharmaceutical composition comprising an mRNA-LNP. Analysis of the test samples by the assay described herein provides information about the relative potency of the samples. In embodiments of the methods described herein, the test sample is analyzed at a fraction of full strength, e.g., after dilution.

“Reference Sample” or “Reference Standard” as used herein describes a standard or control sample relative to which a test sample is compared. As used herein, the reference standard of a composition comprising an mRNA encapsulated in a LNP is designated as having a potency of 100%, and is used to calculate the relative potency of a test article or test sample. As a non-limiting example, a reference sample may be a sample which has been shown to exhibit good expression of the polypeptide encoded by the mRNA in animal models and/or a therapeutic effect in vivo. Typically, as understood by those skilled in the art, a reference sample is determined or characterized under comparable conditions or circumstances to those under assessment (e.g., to the test sample). In embodiments of the methods described herein, the reference sample is analyzed at a fraction of full strength, e.g., after dilution. As a non-limiting example, the reference sample (and the test sample) may be analyzed using a 2-fold serial dilution.

“mRNA” or “Messenger RNA” as used herein refers to a nucleotide polymer comprising predominantly ribonucleotides and encoding a polypeptide or protein. mRNA typically comprises from 5′ to 3′, a cap, an untranslated region, an open reading frame encoding a protein or polypeptide, a 3′ untranslated region and a 3′ poly(a) tail. In some embodiments, the mRNA may comprise one or more modified or non-natural nucleotide residues.

“Lipid Nanoparticles” or “LNP” as used herein refers to any lipid composition that forms a particle, including but not limited to liposomes or vesicles, having a length or width measurement (e.g., a maximum length or width measurement) between 10 and 1000 nanometers. In embodiments of the method used herein, the LNP is used to deliver a therapeutic product, such as a mRNA. In some embodiments, the lipid composition includes a lipid-defined interior volume in which a therapeutic agent is encapsulated. In some embodiments, a lipid nanoparticle includes an interior volume that is encapsulated by amphipathic lipid bilayers (e.g., single; unilamellar or multiple; multilamellar). In some embodiments, a lipid nanoparticle forms a lipid aggregate in which the encapsulated therapeutic agent is contained within a relatively disordered lipid mixture. In some embodiments, a lipid nanoparticle forms a lipid aggregate in which the encapsulated therapeutic agent is contained within a relatively ordered lipid mixture, forming non-lamellar structures (e.g. micelle, hexagonal, etc.). The methods described herein comprises LNPs which have the mRNA encapsulated therein. In embodiments of the method described herein, the LNP comprises a cationic lipid, a PEG-lipid, a sterol, and a non-cationic lipid. In other embodiments, the LNP comprises a cationic lipid, a PEG-lipid, cholesterol, and a phospholipid.

“mRNA-LNP” as used herein refers to an mRNA that is encapsulated within a lipid nanoparticle. In some embodiments, the mRNA-LNP is an LNP encapsulating an mRNA encoding an RSV peptide, as described, for example, in WO 2017/070622 (PCT/US2016/058321), WO 2018/170260 (PCT/US2018/022630) or WO 2019/148101 (PCT/US2019/015412). In some embodiments, the mRNA encodes an RSV F. In other embodiments, the mRNA-LNP is an LNP encapsulating an mRNA encoding a VZV peptide, such as a VZV glycoprotein E, or variants, truncations, or truncated variants thereof, as described, for example in WO/2017/070601 (PCT/US2016/058297).

“Cationic lipid” as used herein refers to a lipid species that carries a net positive charge at a selected pH, such as physiological pH. Those of skill in the art will appreciate that a cationic lipid can be an ionizable lipid, such as an ionizable cationic lipid. Such lipids include, but are not limited to, U.S. Patent Application Publication Nos. US 2008/0085870, US 2008/0057080, US 2009/0263407, US 2009/0285881, US 2010/0055168, US 2010/0055169, US 2010/0063135, US 2010/0076055, US 2010/0099738, US 2010/0104629, 2013/0017239, and US 2016/0361411, International Patent Application Publication No. WO2011/022460 A1; WO2012/040184, WO2011/076807, WO2010/021865, WO 2009/132131, WO2010/042877, WO2010/146740, WO2010/105209, and in U.S. Pat. Nos. 5,208,036, 5,264,618, 5,279,833, 5,283,185, 6,890,557, and 9,669,097. In some embodiments, the cationic lipid is (13Z,16Z)—N,N-dimethyl-3-nonyldocosa-13,16-dien-1-amine; and N,N-dimethyl-1-[(1 S,2R)-2-octylcyclopropyl]heptadecan-8-amine; or a pharmaceutically acceptable salt thereof, or a stereoisomer of any of the foregoing, or any combination of the foregoing

“Plate” or “Cell plate” or “Cell culture plate” means multiwell plate(s). In an embodiment of the present invention, “plate(s)” “cell plate(s)” or “cell culture plate(s)” means multiwell plate(s) that are manufactured with 6, 12, 24, 48, 96, 384 or 1536 sample wells in each plate. In another embodiment, plate(s) are multiwell plate(s) with 24 sample wells or more. In another embodiment, plate(s) are multiwell plate(s) with 48 sample wells or more. In another embodiment, plate(s) are multiwell plate(s) with 96 sample wells or more. In another embodiment, plate(s) are multiwell plate(s) with 384 sample wells or more. In another embodiment, plate(s) are multiwell plate(s) with 1536 sample wells or more. In another embodiment, plate(s) are 96 sample well plate(s). In another embodiment, plate(s) are 384 sample well plate(s). In another embodiment, plate(s) are 1536 sample well plate(s).

In some embodiments, the wells of the plate do not contain a coating. In some embodiments, the wells of the plate are coated. In one embodiment, the wells of the plate are coated with collagen. In another embodiment, the wells are coated with lysine. In a further embodiment, the wells are coated with poly-L-lysine.

The term “seeded” or “seeding” means the addition of cells to a plate with appropriate growth media. The term “seeding density” as used herein is the concentration or number of cells that are added per each well of a multiwell plate to form a monolayer. In some embodiments the seeding density is between about 5×10⁴ cells per well to 1.2×10⁵ cells per well, when the plate is a multiwell plate with 96 wells and which does not contain a coating. In another embodiment, the seeding density is between 1.0×10⁵ cells per well and about 1.2×10⁵ cells per well, when the plate is a multiwell plate with 96 wells and which does not contain a coating. In a further embodiment, the seeding density is about 1.2×10⁵ cells per well, when the plate is a multiwell plate with 96 wells and which does not contain a coating.

In another embodiment, the seeding density is up to 35,000 cells per well, when the plate is a multiwell plate with 96 wells and contains a coating. In another embodiment, the seeding density is between 15,000 to 35,000 cells per well, when the plate is a multiwell plate with 96 wells and contains a coating. In a further embodiment, the seeding density is between 20,000 cells per well to 30,000 cells per well, when the plate is a multiwell plate with 96 wells and contains a coating. In another embodiment, the seeding density is about 20,000 cells per well, when the plate is a multiwell plate with 96 wells and contains a coating. In another embodiment, the seeding density is about 30,000 cells per well, when the plate is a multiwell plate with 96 wells and contains a coating.

The term “confluency” means the percentage of the surface of a cell culture plate that is covered by adherent cells. In embodiments of the invention, the cells are seeded and grown to a confluency such that a monolayer of cells is formed in the sample well of the plate prior to the transfection of the cells with the mRNA-LNP.

### Example 1

### Selection of Cell Line for Cell Base Potency Assay

In-vivo transfection and subsequent protein translation of mRNA by LNPs containing mRNA is a multi-step process which requires cell-entry of the LNP by endocytosis, followed by escape of the mRNA from the endosomal membrane vesicle into the cytosol, binding of the mRNA to ribosomes and subsequent translation of protein. Selection of a cell type as a substrate for an in-vitro assay to measure mRNA-LNP potency stability is an important first step in the development of an in vitro cell-based potency assay. Such cell type must be able to uptake the LNP and subsequently express the mRNA for such cell type to serve as a substrate for an in vitro cell based potency assay. The following cell substrate attributes are desirable for an in-vitro potency assay:
1) Utilize endocytosis pathways shown to be important in uptake of LNPs containing mRNA, particularly LDL-R/ApoE mediated endocytosis;
2) An immortal cell-line and not primary cell lines;
3) A wide sensitivity to discern differences in lot to lot potency or stability;
4) Sensitivity to biologically relevant properties of LNPs, such as size of the LNP and mRNA content.

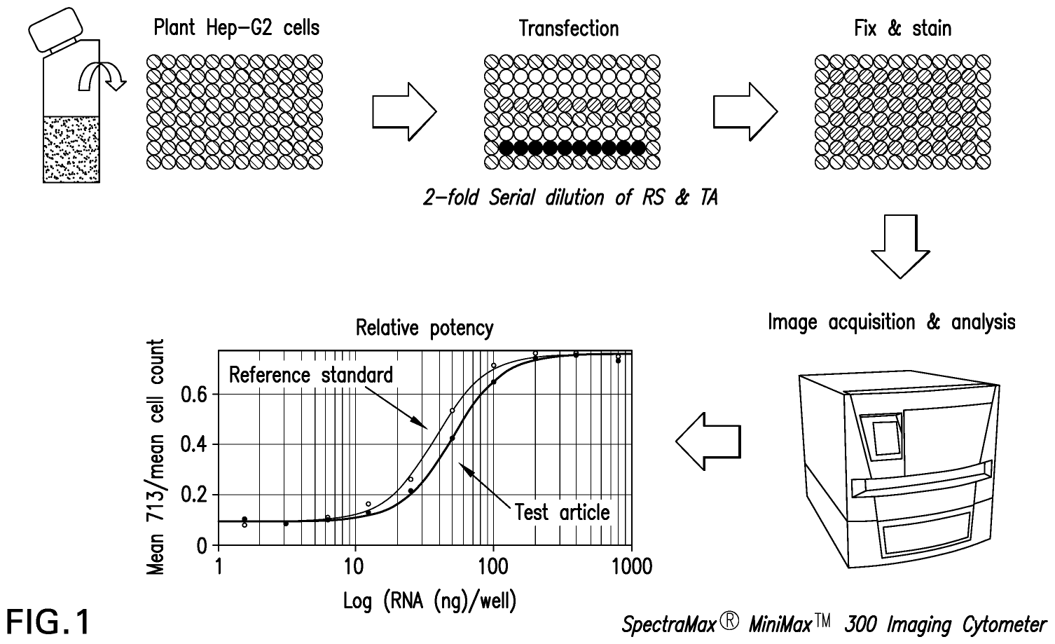

There are several cell-lines and cell types available which could serve as an assay substrate, with each cell type possessing a unique phenotype. The different cell lines were sourced from ATCC and cultured according to ATCC's guidance. The cells were then seeded in 96-well plates and allowed to incubate for 1 day prior to carrying out the transfection procedure as described in the examples that follow. A general schematic of the cell-based potency assay described herein is set forth in FIG. 1.

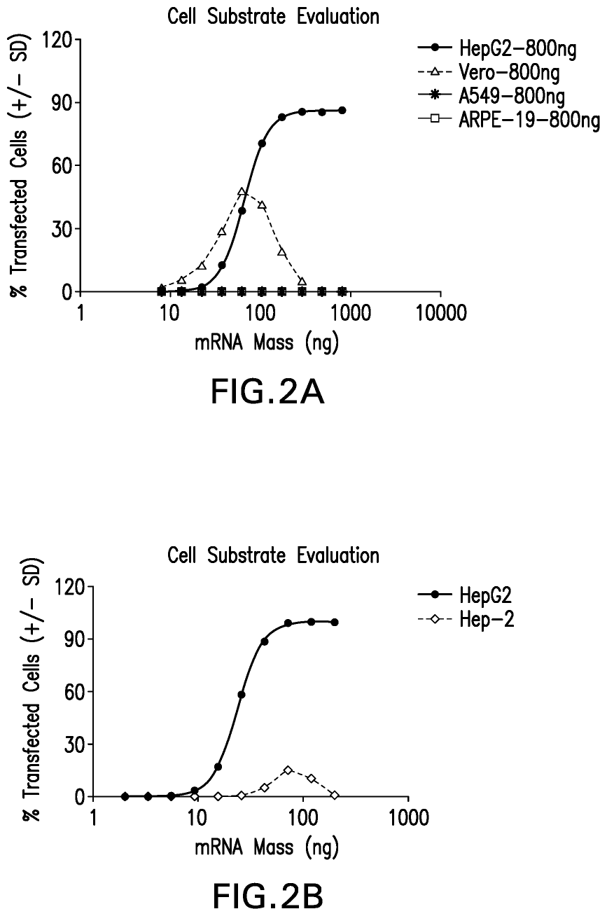

Transfection and protein translation for an mRNA encapsulated in an LNP was found to be highly variable between cell-types, as shown in the table below and in FIGS. 2A-2D. Minimal to no protein expression was observed for some cell lines, while for other cell lines protein expression was observed but there was either also a hook effect observed or the sensitivity of the dose-response curve, as determined by the midpoint of the four-parameter fit, was decreased. A hook effect is a phenomenon where a decreasing response or expression is observed with increasing dose. A hook effect can be caused by various factors such as 1) depletion of essential nutrients for cellular uptake or intracellular processing or 2) toxicity of the material or matrix components on the cells. The different cell lines tested were found to be sensitive to different lots of fetal bovine serum (FBS). This sensitivity to lots of serum was not observed with the HepG2 cells and the cells were found to be more robust for this purpose. As summarized, the data illustrates that the HepG2 cell line is the optimal substrate and best fits the desired attributes described above. However, Vero cells, HeLa cells, and RD cells also displayed high transfection efficiency, with Vero and Hela cells showing a pronounced hook effect in the absence of ApoE spiked into media at transfection (at 4 μg/mL). The addition of ApoE to the media for Vero and HeLa cells eliminated the hook effect and allowed complete transfection by both cell-lines. Similarly, while the transfection of HEp-2 cells was minimal without additional ApoE, full transfection was achieved with the addition of ApoE (data not shown). RD cells can be completely transfected without additional ApoE. However, addition of ApoE to the media did improve transfection, as seen by a decrease in EC50 values when ApoE was added.

TABLE 1

| Cell Name | Description | Cell Type | Data Summary |
|---|---|---|---|
| HepG2 | Human Liver | Epithelial-like | High sensitivity and expression |
| | Carcinoma | (carcinoma) | of mRNA |
| | | | Abundance of LDL receptors |
| | | | Multiple Entry Pathways to |
| | | | incorporate LNP, including |
| | | | CME and macropinocytosis |
| A549 | Human Lung | Epithelial | No uptake of LNP or |
| | Carcinoma | | expression of mRNA |
| Caco-2 | Human Colon | Epithelial-like | No uptake of LNP or |
| | Adenocarcinoma | (carcinoma) | expression of mRNA |
| ARPE-19 | Human Retina | Epithelial | No uptake of LNP or |
| | | | expression of mRNA |
| HEp-2 | Human (HeLa | Epithelial-like | Minimal uptake of LNP or |
| | contaminant) | (carcinoma) | mRNA expression |
| | | | Significant hook effect |
| | | | observed |
| Vero | African Green | Epithelial | Some uptake of LNP and |
| | Monkey Kidney | | mRNA expression |
| | | | Significant hook effect |
| | | | observed |
| HeLa | Human Cervical | Epithelial-like | Some uptake of LNP and |
| | Adenocarcinoma | (carcinoma) | mRNA expression |
| | | | Significant hook effect |
| | | | observed |
| RAW 264.7 | Mouse Leukemia | Macrophage | No uptake of LNP or |
| | virus-induced tumor | (Leukemia) | expression of mRNA |
| RD | Human Muscle | Spindle, | Full dose-response curves |
| | (Rhabdomyosarcoma) | multi-nucleated | 1.5x lower sensitivity (EC50) |
| | | | compared to HepG2 |

### Example 2

### In-Vitro Cell Based Potency Assay

### General Assay Conditions:

A general schematic of the assay is set forth in FIG. 1. First, cells (e.g., HepG2 cells (ATCC)) are plated in a 96 well culture plate with EMEM (EMEM with L-Glutamine from ATCC) plus 2% FBS (heat inactivated, ATCC) and placed in an incubator (37° C. and 5% CO₂) for 1 day (24 hours) prior to transfection. The cells are grown to a confluency such that a monolayer of cells if formed (˜ 70-85% confluency). LNPs encapsulating mRNA are diluted with a diluent (e.g., Opti-MEM® Medium, Life Technologies), such that the highest concentration of RNA used is 800 ng/well. The HepG2 cell monolayers are transfected with the LNP mRNA samples by adding the diluted LNP mRNA to the HepG2 monolayers and incubating at 37° C., 5% CO₂ for 16-18 hours. After transfection, the media is removed, and the cells are fixed with 3.7% formaldehyde fixing solution and permeabilized by washing the plates three times with 100 μL/well of PBS/1% Titron X-100. 50 μL/well of diluted primary antibody (1 μg/mL; specific for the protein encoded by the mRNA) is added to each well and plates are incubated for 1-3 hours with moderate shaking. Plates are then washed three times with 100 μL/well of PBS/0.05% Tween® 20. The secondary antibody (IRDye 680 RD goat α-human; diluted 1:100 from 1 mg/mL stock to 10 μg/mL) is subsequently added and plates are incubated at room temperature for at least 2 hours with moderate shaking. Plates are washed three times with 100 μL/well of PBS. The secondary antibody is detected by scanning the plates using an imaging device, e.g., SpectraMax® MiniMax™ 300 Imaging Cytometer (Molecular Devices)

For reference samples and each test sample, the percentage of cells transfected is graphed against the mRNA dose and a four-parameter logistic (4-PL) regression is used to determine a model for the dose-response relationship. The four parameters used to determine a 4-PL model are the top and bottom asymptotes, slope, and EC50 of the curve. The EC50 value is the dose at which the value of the response is halfway between the background (bottom asymptote) and the maximum response (top asymptote). Parallelism is evaluated to assess similarity between the reference and test article with only a differing EC50 value and the other three parameters being similar. To assess parallelism between the reference and the test article, a full 4-PL model is determined for reference and the test article individually and a ratio of the slopes is evaluated. Next, a reduced pairwise 4-PL is modeled with a common top asymptote, bottom asymptote, and slope but with a varying EC50 parameter between the reference and each test article. Finally, the relative potency of the test article is determined by comparing the EC50 of the reference sample with the EC 50 of the test article to obtain a relative potency value using the following formula:

Relative Potency=(EC50 Ref/EC50_{TA})*100.

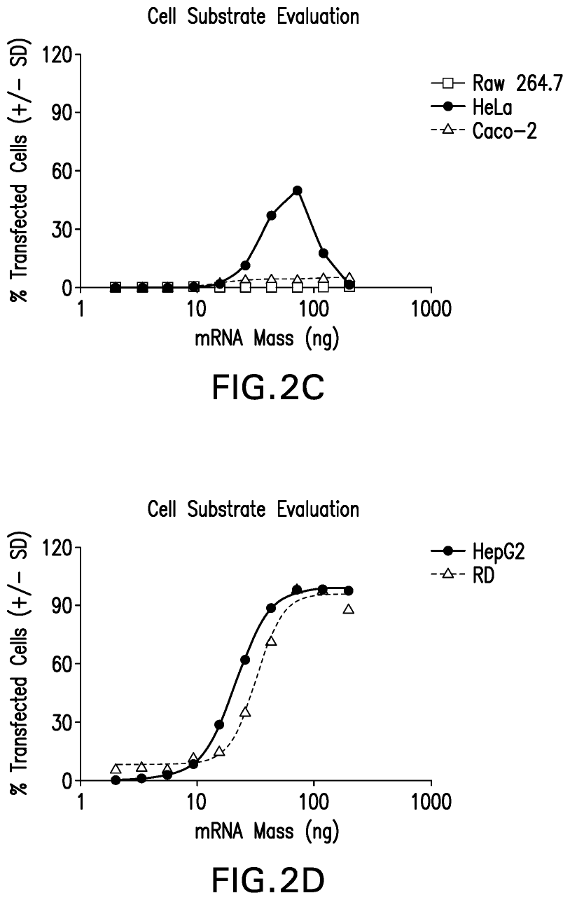

In this example, the potency of an LNP sample encapsulating an mRNA encoding an RSV F prefusion protein was determined. The general assay conditions were as described above. Four cell lines were evaluated: HepG2, Hep-2, Vero, and ARPE-19 at 75% confluency. As shown in FIGS. 3A and 3B, the HepG2 cells performed the best as the level of polypeptide expressed by the mRNA in the Hep-G2 cells was much higher compared to other cell lines in which almost no uptake and expression was observed.

Seeding densities in the range of 5.0×10⁴ to 1.2×10⁵ were evaluated. Seeding densities in the range of 5×10⁴ and 8×10⁴ require 2 days of incubation to reach optimal confluency, whereas seeding densities in the range of 1.0×10⁵ and 1.2×10⁵ needed 1-day incubation to reach optimal confluency and generate a full dose-response curve (data not shown). Thus, seeding densities in the range of 1.2×10⁵ and 1.0×10⁵ are more efficient in data acquisition as they require less incubation time, allowing for a more rapid potency assay.

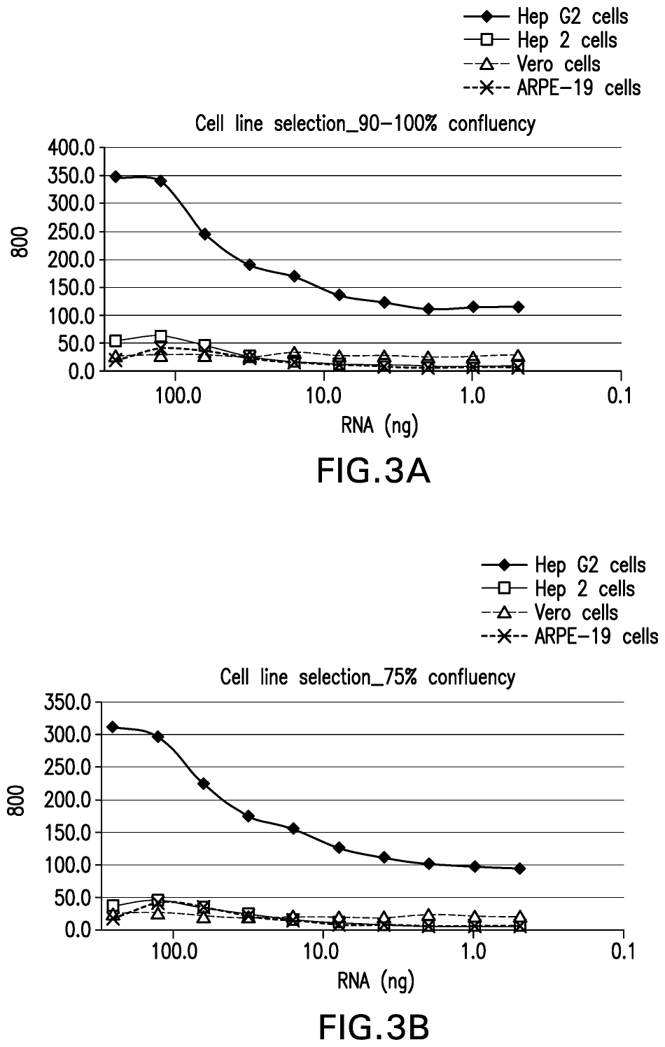

The duration of transfection in the range of 5 to 48 hours was evaluated. As shown in FIGS. 4A, 4B and 4C, the transfection rate increased from 5 hours to 7 hours, after which the transfection rate reached to a plateau. At 16 hours, the transfection rate is only slightly higher than at 7 hours. At 48 hours, the data still showed a full curve, although the transfection rate is lower (data not shown).

### Example 3

### In-Vitro Cell Based Potency Assay Using Coated Plates

The use of coated versus un-coated cell culture plates was also evaluated. Specifically, collagen coated plates and poly-L-lysine coated plates were also evaluated. Collagen coated plates allow the HepG2 cells to spread out and form a monolayer rather than balling up or clumping in cell culture treated plates. A monolayer formation of cells is essential for uptake kinetics and improves sensitivity and precision of the assay.

HepG2 cells were first plated in a 96-well collagen coated plates with EMEM (EMEM with L-Glutamine from ATCC) plus 2% FBS (heat inactivated, ATCC) and placed in an incubator (37° C. and 5% CO₂) for 1 day (22±6 hours) prior to transfection. The cells were grown to a confluency such that a monolayer of cells is formed (>70% confluency). LNPs encapsulating mRNA were diluted with a diluent (e.g., Opti-MEM® Medium, Life Technologies), such that the highest concentration of mRNA used is 200 ng/well. The spent media is removed from the HepG2 cell monolayers and replenished with fresh EMEM plus 2% FBS media. The cells are transfected with the LNP mRNA samples by adding the diluted LNP mRNA to the HepG2 monolayers and incubating at 37° C., 5% CO₂ for 16±2 hours. Following transfection, the media was removed, and the cells were fixed with 3.7% formaldehyde fixing solution. Cells were permeabilized by incubating at ambient temperature in 100 μL/well of PBS/0.5% Titron X-100. 50 μL/well of diluted primary antibody (2 μg/mL; specific for the protein encoded by the mRNA) was added to each well and plates were incubated for 1-3 hours. Plates were then washed two times with 100 μL/well of PBS/0.1% Tween® 20. The secondary antibody was added (IRDye 680 RD goat α-human or Alexa Fluor 488 goat α-human; diluted 1:100 from 1 mg/mL stock or 1:200 from 2 mg/mL stock, respectively, to 10 μg/mL). Plates were incubated again at room temperature for at least 30 minutes and then washed two times with 100 μL/well of PBS/0.1% Tween-20. Plates were scanned using an imaging device, e.g., SpectraMax® MiniMax™ 300 or Biotek Cytation3/Cytation5 Imaging Cytometer. The relative potency of the LNP mRNA samples was determined by pairwise four parameter logistic analysis of the protein expression of the mRNA to that of a reference standard.

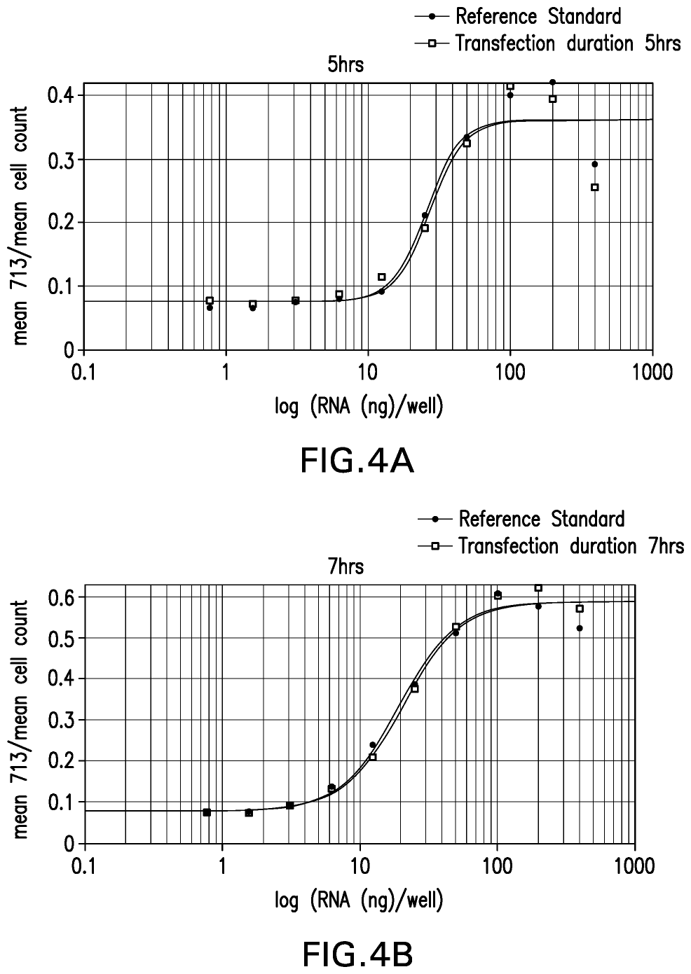

For coated plates, seeding densities in the range of 15,000 to 35,000 cpw (cells per well) were evaluated and incubated for 22±6 hours. The seeding densities of 20,000 cpw to 30,000 cpw were found to be the most optimal since >75% confluency was observed at these densities while not over-crowding the well. However, as shown in FIG. 5, there was no difference observed between any of the seeding densities that were evaluated, and all seeding densities were found to be acceptable for the assay.

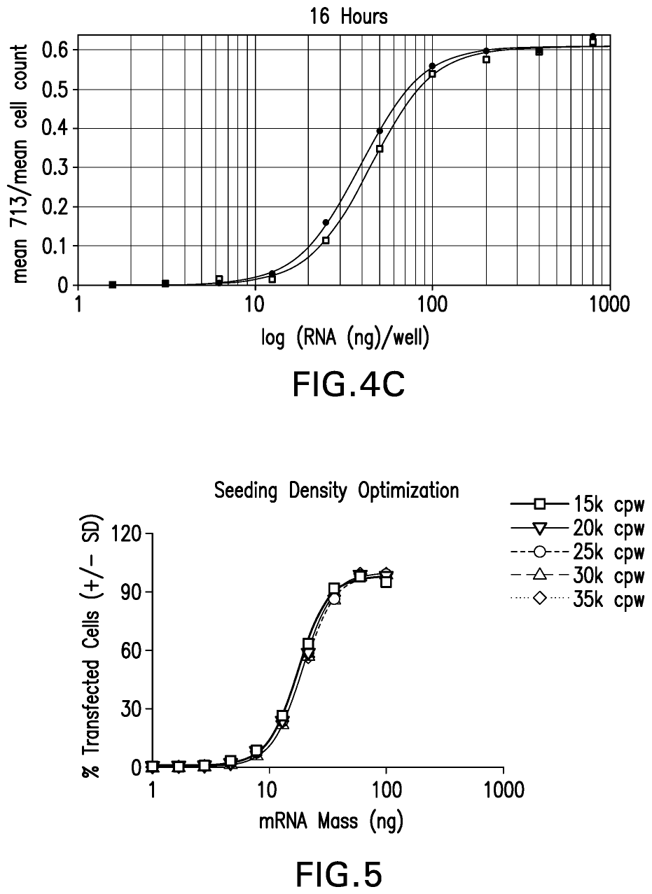

Transfection time ranging from 4 hours to 16 hours was evaluated. As shown in FIG. 6, at least 6 hours was required to achieve a full dose-response curve of protein expression. It was observed that increasing the incubation duration increased the sensitivity as measured by midpoint of the 4-PL curve. A 16-hour duration was preferred for higher sensitivity in the assay; however, a 6 hour or greater duration is acceptable in the assay.

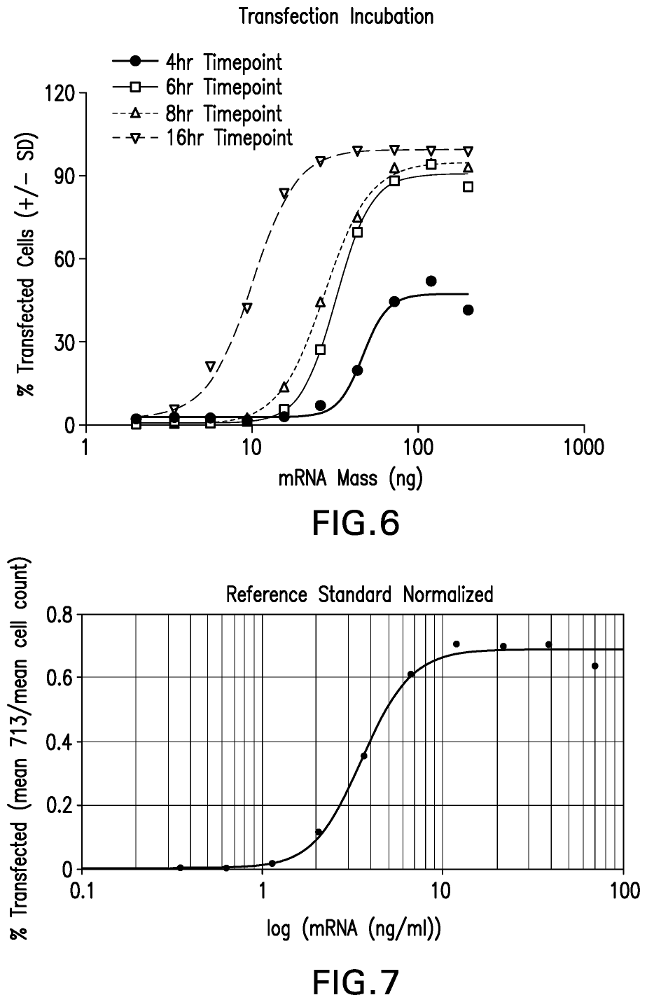

Poly-L-lysine coated plates were also evaluated. Due to the synthetic nature of Poly-L-lysine, it cannot introduce any animal derived impurities to the assay. The general assay conditions were similar as described above, the results of one example were shown in FIG. 7, with cell seeding density 25,000 cpw, the highest concentration of mRNA used 70 ng/well, the concentration of the primary Abs 1 μg/ml and the media on the plate was not exchanged prior transfection and transfection duration 16 hours.

1. A method for determining the relative in-vitro potency of a composition comprising an mRNA encapsulated in a lipid nanoparticle (LNP), the method comprising:
(i) transfecting a population of cells with a test sample of the composition, wherein the cells are selected from Vero cells, HeLa cells, RD cells, Hep-2 cells and Hep-G2 cells;
(ii) transfecting a different population of cells with a reference sample of the composition, wherein the cells are the same cell type as selected for step (i);
(iii) detecting the amount of expression of a polypeptide encoded by the mRNA in the transfected cells of step (i) and step (ii); and
(iv) comparing the amount of expression of the polypeptide determined for the test sample in step (iii) with the amount of expression of the polypeptide determined for the reference sample in step (iii) thereby determining the relative in vitro potency of the composition.

2. The method of claim 1, wherein detecting the expression of the polypeptide in step (iii) comprises contacting the transfected cells with a first antibody specific for the polypeptide encoded by the mRNA and subsequently with a second, labeled antibody which is specific for the first antibody.

3. The method of claim 2, further comprising detecting the second, labeled antibody.

4-5. (canceled)

6. The method of claim 1, wherein ApoE is added during step (i).

7. (canceled)

8. The method of claim 1, wherein the cells are Hep-G2 or RD cells.

9. The method of claim 8, wherein no ApoE is added during step (i).

10. The method of claim 1, wherein the LNP comprises a cationic lipid, a sterol, a non-cationic lipid, and a pegylated-lipid.

11. The method of claim 1, further comprising seeding the cells on a cell culture plate comprising at least 12, 24, 48, 96 or 384 wells prior to transfecting the cells.

12. The method of claim 11, wherein the wells of the cell culture plate do not contain a coating.

13. (canceled)

14. The method of claim 11, wherein the wells of the cell culture plate coated with collagen or lysine.

15. The method of claim 11, wherein the seeded cells are grown to a confluency in which a monolayer of cells is formed.

16. The method of claim 15, wherein the seeded cells are grown for about 16 to about 32 hours prior to transfecting.

17. (canceled)

18. The method of claim 11, wherein each well of the cell culture plate is seeded with about 1.1×10⁵ cells to about 1.4×10⁵ cells per well when the wells of the cell culture plates are not coated.

19. (canceled)

20. The method of claim 11, wherein the cells are seeded in a 96 well plate at a density of 15,000 cells per well to 35,000 cells per well.

21. The method of claim 20, wherein each well of the cell culture plate is seeded with 20,000 cells per well to 30,000 cells per well.

22. (canceled)

23. The method of claim 21, wherein the transfecting process of step (i) occurs at 35-39° C. with 4-6% CO₂ for at least 4 hours.

24. The method of claim 1, comprising generating a dose response curve for the test sample and the reference sample and determining the EC50 of the test sample and the reference sample.

25. The method of claim 24, wherein the relative potency is calculated as a percentage of the reference standard EC50 using the formula
(EC50 reference standard/EC50 test sample)*100

26. A process for releasing or accepting a batch of a pharmaceutical composition comprising an mRNA encapsulated in an LNP, comprising
(i) determining the relative in vitro potency of a test sample of the pharmaceutical composition from the batch according to the method of claim 1; and
(ii) releasing further pharmaceutical compositions from the batch for in vivo use if the results of step (i) indicate an acceptable relative in vitro potency value.

27. The process of claim 26, wherein the relative in vitro potency value is calculated by generating a dose response curve for the test sample and the reference sample and determining the EC50 of the test sample and reference sample, and wherein the relative in vitro potency value is calculated using the formula
(EC50 reference standard/EC50 test sample)*100.

28. (canceled)

29. The process of claim 27, wherein the acceptable relative in vitro potency value is calculated to be between 50% and 200%.
